(19) United States
(12) Patent Application Publication
(10) Pub. No.: US 2014/0005597 A1
(43) Pub. Date: Jan. 2, 2014

(54) KIT AND METHOD FOR USE IN ADMINISTERING THERAPEUTIC BOTULINUM TOXIN (BOTOX)

(71) Applicant: David P. Jankowski, Birmingham, MI (US)

(72) Inventor: David P. Jankowski, Birmingham, MI (US)

(73) Assignee: Compassion Physicians Associates, P.C., Birmingham, MI (US)

(21) Appl. No.: 13/874,836

(22) Filed: May 1, 2013

Related U.S. Application Data
(60) Provisional application No. 61/640,851, filed on May 1, 2012.

(51) Int. Cl. A61M 5/00

(52) U.S. Cl. CPC A61M 5/002
USPC 604/87

(57) ABSTRACT

A preassembled kit organizes the administration of therapeutic botulinum toxin by providing all of the important ingredients necessary for the procedure, including a vial of botulinum toxin preserved on dry ice. The kit also includes saline solution sufficient to dilute the botulinum toxin; a syringe to inject the saline solution into the vial of botulinum toxin; one or more needles to administer the diluted botulinum toxin; and other items associated with the procedure. Such items may include a plurality of absorbent pads, a patient gown, and clinician gloves. Particularly for use in the administration of therapeutic botulinum toxin, the kit may further include at least one radiolucent electrode, and at least one needle adapted for connection to a nerve stimulator.

### REFERENCE TO RELATED APPLICATION

This application claims priority from U.S. Provisional Patent Application Ser. No. 61/640,851, filed May 1, 2012, the entire content of which is incorporated herein by reference.

FIELD OF THE INVENTION

This invention relates generally to the administration of botulinum toxin and, in particular, to a kit containing all of the important ingredients necessary for the procedure, including a vial of botulinum toxin preserved on dry ice.

### BACKGROUND OF THE INVENTION

Botulinum toxin (also known as botulin toxin, botulinum neurotoxin, and “Botox®”) is one of the most potent naturally occurring neurotoxins. By preventing synaptic transmission or release of acetylcholine across the neuromuscular junction, botulinum toxin blocks signals that normally would cause muscle spasms or contractions, resulting in muscle paralysis.

Most people think of botulinum toxin as a treatment for reducing the signs of aging. In cosmetic applications, a small botulinum toxin injection can prevent the development of wrinkles by paralyzing facial muscles. Botulinum toxin injection is now the most common cosmetic operation in the United States.

In addition to its cosmetic applications, however, botulinum toxin is also used in the treatment of spasms and dystonias by weakening the muscles involved. Conditions that may be treated with botulinum tennis elbow, chronic anal fissure, pain attributed to mastectomy and hemorrhoidectomy, headaches (including migraine), piriformis syndrome, facial pain, myofascial pain, temporomandibular joint syndrome (TMJ), low back pain, chronic prostatic pain, and whiplash.

Botulinum toxin is typically administered via injection to the area in need of treatment. The injection typically involves reconstituting a previously lyophilized sample of botulinum toxin using saline or some other pharmaceutically acceptable diluent. The reconstituted botulinum toxin solution is then drawn into a syringe, where it remains until injection into the patient. The sequence of steps required for preparation and subsequent administration of the botulinum toxin is relatively safe for the clinician, as the reconstituted botulinum toxin solution is contained in either the syringe or a vial until administration.

Administration of botulinum toxin requires an array of ancillary medical supplies and equipment in addition to the botulinum toxin itself. Such products must be drawn together form different sources or locations prior to the procedure. Therapeutic Botox comes in a vacuum sealed vial in powder form, and the vial of Botox must be preserved on dry ice at all times until it is diluted with 0.9% sodium chloride. Typically, an EMG/nerve stimulator is required to assist in proper muscle localization for injection for treatment of cervical dystonia and chronic migraines. Given the disparate materials and equipment involved, the treatment of painful muscle syndromes botulinum toxin administration can be a difficult and frustrating task for any clinician.

### SUMMARY OF THE INVENTION

This invention organizes the administration of therapeutic botulinum toxin by providing a kit with all of the important ingredients necessary for the procedure, including a vial of botulinum toxin preserved on dry ice. The kit also includes saline solution sufficient to dilute the botulinum toxin; a syringe to inject the saline solution into the vial of botulinum toxin; one or more needles to administer the diluted botulinum toxin; and other items associated with the procedure. Such items may include a plurality of absorbent pads, a patient gown, and clinician gloves. Particularly for use in the administration of therapeutic botulinum toxin, the kit may further include at least one radiolucent electrode, and at least one needle adapted for connection to a nerve stimulator with the cervical dystonia kit.

### DETAILED DESCRIPTION OF THE INVENTION

This invention improves upon the administration of therapeutic botulinum toxin by providing a kit containing all the necessary medical articles and equipment, thereby alleviating the clinician from have to locate all of the necessary items or, worse, beginning the procedure only to discover that some desired items are unavailable.

In the preferred embodiment, the kit would include all of the items listed below, though quantities and source of supply (i.e., certain brands) may vary. In alternative embodiments, certain items may be left out, such as the electrodes, if it is known in advance that nerve stimulation will not be used. In terms of the nerve stimulator, a Stimuplex HNS 12 Nerve Stimulator is applicable to the invention, though this would not be supplied as part of the kit.

Kit Contents for Cervical Dystonia

| | |
|---|---|
| Allergan Botox - Therapeutic 200 unit vial preserved on Dry Ice | 1 |
| 18G × 1½ needle with 5 cc syringe | 1 |
| 10 mL single dose 0.9% preservative free sodium chloride | 1 |
| Alcohol prep pads | 15, +/− |
| Gauze 2 × 2 | 10, +/− |
| 850 Foam ECG Radiolucent Electrodes | 1 |
| Stimuplex A 0.70 × 50 mm 25 × 2″ needle | 1 |
| Sterile Gloves | 1 |
| 27G × 1½ needle with 5 cc syringe | 1 |

Kit Contents for Migraines

| | |
|---|---|
| Allergan Botox - Therapueutic 200 unit vial preserved | 1 |
| on Dry Ice | |
| 18G × 1½ needle with 5 cc syringe | 1 |
| 10 mL single dose 0.9% preservative free sodium chloride | 1 |
| Alcohol prep pads | 15, +/− |
| Gauze 2 × 2 | 10, +/− |
| Sterile Gloves | 1 |
| 25G or 27G × 1½ needle with 5 cc syringe | 1 |

### Preparation for the Botox Injection Using the Kit

1. Open Botox vial—remove cap, use one alcohol prep pad to clean top of vial

2. Open vial of 10 ml Single dose 0.9% preservative free sodium chloride and clean with alcohol prep pad

3. Use 18G×1½ needle with 5 cc syringe and draw up 3-4 cc of 0.9% sodium chloride preservative free

4. Inject saline into Botox vial without removing needle from vial.

5. Twirl bottle to dilute all powder.

6. Leave needle in Botox vial and undo syringe to release vacuum

7. Recap syringe and draw up all Botox into syringe

8. Use second 18G×1½ needle with 5 cc syringe to draw up 2-3 cc of preservative free saline to flush out medication

### The Procedure Using the Kit

1. Hand gown to patient and allow to dress with opening of gown to back

2. Medical Assistant will place Electrode on patient

3. Connect stimulator to syringe containing Botox

4. Connect Stimuplex 22G×2″ needle to other end of nerve stimulator

5. Medical Assistant wears gloves and assists doctor using sterile technique to wear sterile gloves.

6. Medical Assistant uses alcohol prep pad to clean area(s) of injection

7. Medical Assistant uncaps Stimuplex needle

8. Doctor will then flush the Botox throughout the needle until the medication reaches the tip

9. Ready to inject muscles.

1. A kit for use in the administration of therapeutic botulinum toxin, comprising:
a vial of botulinum toxin preserved on dry ice;
saline solution sufficient to dilute the botulinum toxin;
a syringe to inject the saline solution into the vial of botulinum toxin;
one or more needles to administer the diluted botulinum toxin; and
other items associated with the procedure, including one or more of the following:
a plurality of absorbent pads and
clinician gloves.

2. The kit of claim 1, further including:
at least one radiolucent electrode; and
at least one needle adapted for connection to a nerve stimulator.
